(19) United States
(12) Patent Application Publication
(10) Pub. No.: US 2006/0008451 A1
(43) Pub. Date: Jan. 12, 2006

(54) In vivo methods for effecting tissue specific differentiation of embryonic stem cells

(75) Inventors: Jose Cibelli, East Lansing, MI (US); Naoki Tsunekawa, Okemos, MI (US); Akiko Yabuuchi, East Lancing, MI (US)

Correspondence Address: DUANE MORRIS LLP, 1667 K. STREET, N.W., SUITE 700, WASHINGTON, DC, 20006-1608, US

(73) Assignee: Michigan State University, East Lansing, MI (US)

(21) Appl. No.: 11/174,603

(22) Filed: Jul. 6, 2005

Related U.S. Application Data
(60) Provisional application No. 60/585,100, filed on Jul. 6, 2004.

(51) Int. Cl. A61K 48/00; C12N 5/08

(52) U.S. Cl. 424/93.21; 435/366

(57) ABSTRACT

Methods for producing derived differentiated cells from chimeric animals, fetuses and embryos derived from non-human chimeric embryos are provided. These non-human chimeric embryos are obtained by introduction of at least one transgenic ES or ES-like cell into a recipient non-human embryo, e.g., blastocyst stage embryo, wherein said ES or ES-like cell comprises DNA constructs that contain markers and promoters which allow for selective survival of a desired differentiated cell, tissue organ and ablation of other cells that originate from said transgenic ES or ES-like cells.

### FIELD OF THE INVENTION

This invention relates generally to the field of cell biology, embryology, and embryonic stem cells. More particularly, the invention provides a method for obtaining desired differentiated cells from embryonic stem cells, preferably primate embryonic stem cells, by controlled differentiation by a method which combines recombinant and embryo transfer techniques using embryos obtained by conventional, parthenogenic or nuclear transfer methods. Even more specifically, the invention provides methods for facilitating controlled (tissue specific) differentiation of embryonic stem (ES) cells in vivo by: (i) the introduction of recombinant DNA sequences into donor ES cells that faciliate the selective survival of desired (target) differentiated donor tissue cells originating therefrom and the ablation of non-desired (non-target) donor tissue cells when the resultant transgenic embryonic stem cells are introduced into a non-human embryo recipient of the same or different species origin as the transgenic ES cells and the resultant chimeric embryo is implanted into and permitted to differentiate in the uterus of a suitable non-human recipient animal. The resultant chimeric embryo upon maturation produces a chimeric fetus or chimeric animal, e.g., a murine which contains the desired donor (e.g., primate) differentiated cells, tissues or organs. These differentiated cells, tissues or organs are recoverable therefrom and may be used e.g., in cell and genetic therapies.

### BACKGROUND OF THE INVENTION

Precursor cells have become a significant interest in medical research. Many tissues in the body have a back-up reservoir of precursors that can replace cells that are senescent or become damaged by injury or disease. Along those lines, considerable effort has been made recently to isolate procursors of a number of different tissues for use in regenerative medicine.

Over the past several years, considerable interest has been generated by the development of different types of embryonic stem cells, most especially non-human primate and human embryonic stem cells. These cells are thought to have the potential to differentiate into all tissue cell types. Early work on embryonic stem cells was done in mice. Mouse stem cells can be isolated from both early embryonic cells and germinal tissues. Additionally, embryonic stem cells of different types can be isolated from nuclear transfer and from parthenogenically derived embryos. For example, embryonic stem-like cells have been isolated from primate (cyomulgus) embryos derived from parthenogenically activated primate oocytes. (See published U.S. 20040014206, by Robl et al., published Jan. 22, 2004 which discloses the production of pluripotent cells from parthenogenically activated embryos, which application is incorporated by reference in its entirety herein).

Desirable characteristics of embryonic stem cells include their pluripotency, capability to proliferate in vitro in an undifferentiated state indefinitely (immortal), their ability to retain a normal karyotype and to retain their potential to differentiate to derivatives of all three embryonic germ layers (endoderm, mesoderm and ectoderm).

Development of non-human primate and human pluripotent stem cell preparations is considerably less advanced than work with mouse cells. Thomson et al. propagated pluripotent stem cells from lower primates (U.S. Pat. No. 5,843,780; Proc. Natl. Acad. Sci. USA 92:7844, 1995), and then from humans (Science 282:114, 1998). Gearhart and coworkers derived human embryonic germ (hEG) cell lines from fetal gonadal tissue (Shamblott et al., Proc. Natl. Acad. Sci. USA 95:13726, 1998; and U.S. Pat. No. 6,090,622).

Both hES and hEG cells have the long-sought characteristics of pluripotent stem cells: they are capable of being grown in vitro without differentiating, they have a normal karyotype, and they remain capable of producing a number of different cell types. Clonally derived human embryonic stem cell lines maintain pluripotency and proliferative potential for prolonged periods in culture (Amit et al., Dev. Biol. 227:271, 2000). These cells hold considerable promise for use in human therapy, acting as a reservoir for regeneration of almost any tissue compromised by genetic abnormality, trauma, or a disease condition.

International Patent Publication WO 99/20741 (Geron Corp.) refers to methods and materials for growing primate-derived primordial stem cells. In one embodiment, a cell culture medium is provided for growing primate-derived primordial stem cells in a substantially undifferentiated state, having a low osmotic pressure and low endotoxin levels. The basic medium is combined with a nutrient serum effective to support the growth of primate-derived primordial stem cells and a substrate of feeder cells or an extracellular matrix component derived from feeder cells. The medium can further include non-essential amino acids, anti-oxidants, growth factors, hormones as well as nucleosides and pyruvate salts.

A significant challenge to the use of stem cells for therapy is the need to develop means for control of the growth and differentiation of ES cells into a desired particular type of tissue required for treatment of each patient as well as the derivation of cells that are genetically and immunologically compatible with a particular patient. Some prior methods and materials relating to these objectives are described below.

U.S. Pat. No. 4,959,313 (M. Taketo, Jackson Labs) teaches the introduction into ES cells of a particular enhancer sequence that controls the expression of a flanking exogenous or recombinant gene from a promoter accompanying the gene that is not normally expressed in undifferentiated cells.

U.S. Pat. No. 5,639,618 (D. A. Gay, Plurion Inc.) proposes a method for isolating a lineage specific stem cell in vitro, in which a pluripotent embryonic stem cell is transfected with a construct in which a lineage-specific genetic element is operably linked to a reporter gene, culturing the cell under conditions where the cell differentiates, and then separation of cells expressing the reporter from other cells by culturing under selective conditions.

U.S. Pat. No. 6,087,168 (Levesque et. al., Cedars Sinai Med. Ctr.) is directed to transdifferentiating epidermal cells into viable neurons useful for both cell therapy and gene therapy. Skin cells are transfected with a neurogenic transcription factor, and cultured in a medium containing an antisense oligonucleotide corresponding to a negative regulator of neuronal differentiation.

International Patent Publication WO 97/32025 (Mclvor et al., U. Minnesota) proposes a method for engrafting drug resistant hematopoietic stem cells. The cells in the graft are augmented by a drug resistance gene (such as methotrexate resistant dihydrofolate reductase), under control of a promoter functional in stem cells. The cells are introduced into a mammal, which is then treated with the methotrexate drug to increase engraftment of transgenic cells relative to nontransgenic cells.

International Patent Publication WO 98/39427 (Stein et al., U. Massachusetts) refers to methods for expressing exogenous genes in differentiated cells such as skeletal tissue. Stem cells (e.g., from bone marrow) are contacted with a nucleic acid in which the gene is linked to an element that controls expression in differentiated cells. Exemplary is the rat osteocalcin promoter.

International Patent Publication WO 99/10535 (Liu et al., Yale U.) proposes a process for studying changes in gene expression in stem cells. A gene expression profile of a stem cell population is prepared, and then compared to a gene expression profile of differentiated cells in order to identify genes that affect or modulate differentiation of pluripotent cells into specific cell lineages.

International Patent Publication WO 99/19469 (Braetscher et al., Biotransplant) refers to a method for growing pluripotent embryonic stem cells from the pig. A selectable marker gene is inserted into the cells so as to be regulated by a control or promoter sequence in the ES cells, exemplified by the porcine OCT-4 promoter.

International Patent Publication WO 00/15764 (Smith et al., U. Edinburgh) refers to propagation and derivation of embryonic stem cells. The cells are cultured in the presence of a compound that selectively inhibits propagation or survival of cells other than ES cells by inhibiting a signaling pathway essential for the differentiated cells to propagate. Exemplary are compounds that inhibit SHP-2, MEK, or the ras/MAPK cascade.

Klug et al. (J. Clin. Invest. 98:216, 1996) propose a strategy for genetically selecting cardiomyocytes from differentiating mouse embryonic stem cells. A fusion gene consisting of the α-cardiac myosin heavy chain promoter and a cDNA encoding aminoglycoside phosphotransferase was stably transfected into the ES cells. The resulting lines were differentiated in vitro and selected using G418. The selected cardiomyocyte cultures were reported to be highly differentiated. When engrafted back into mice, ES-derived cardiomyocyte grafts were detectable as long as 7 weeks after implantation.

Schuldiner et al. (Proc. Natl. Acad. Sci. USA 97:11307, 2000) report the effects of eight growth factors on the differentiation of cells from human embryonic stem cells. After initiating differentiation through embryoid body formation, the cells were cultured in the presence of bFGF, TGF-.beta. 1, activin-A, BMP-4, HGF, EGF, .beta.NGF, or retinoic acid. Each growth factor had a unique effect on the differentiation pathway, but none of the growth factors directed differentiation exclusively to one cell type.

U.S. Pat. No. 6,506,574 (Geron) describes the production of an essentially enriched hepatocyte cell population derived from human pluripotent cells by culture in the presence of a hepatocyte differentiation agent.

U.S. Pat. No. 6,576,464 discloses a system for producing a homogeneous differentiated cell population from a stem cell population by introduction of an effector gene, the transcription of which is under the regulatory control of a transcriptional control element (such as the telomerase reverse transcriptase (TERT) promoter) that causes the gene to be expressed predominantly or exclusively in relatively undifferentiated cells comprised in the population. Expression of the effector gene results in depletion of undifferentiated cells, or results in the expression of a marker that allows the cells to be removed at a later time. Types of effector gene sequences exemplified therein include DNAs encoding a toxin, a protein that induces apoptosis, a cell-surface antigen, and an enzyme that converts a prodrug into a substance that is lethal to a cell. Disclosed applications of the resultant differentiated cell populations include tissue regeneration and non-therapeutic applications such as drug screening.

### OBJECTS OF THE INVENTION

Notwithstanding this earlier research in embryonic stem cells and methods for controlled differentiation thereof, there is a need for new approaches to generate populations of differentiated cells from stem and stem-like cells which are suitable for human administration.

It is an object of the invention to provide transgenic embryonic stem cells (ES cells) or embryonic-like (ES-like) cells that comprise at least the following constructs: (i) a first DNA constrct that contains a regulatory sequence (promoter) that is selectively or preferentially expressed in pluripotent or relatively undifferentiated cells that regulates the expression of at least one selectable and/or detectable marker gene; (ii) a second DNA construct that contains another different regulatory sequence (promoter) which is selectively or preferentially in cells of a desired differentiated cell type or lineage, e.g., immune cells, neural cells, skeletal cells, and the like, wherein such promoter regulates the expression of another different selectable and/or detectable marker gene; and (iii) a third DNA construct that contains a selectable and/or detectable marker gene that is expressed under the control of a constitutive promoter and methods for producing such transgenic stem cells.

It is another object of the invention to provide a non-human embryo that is resistant to at least a first and second detectable or selectable marker, wherein the chimeric embryo contains at least one transgenic ES or ES-like cell that is of a different genotype and/or species relative to the remaining cells of the non-human embryo, said transgenic cell containing at least the following transgenes: (i) a first DNA construct that comprises a regulatory sequence (promoter) selectively or predominantly expressed in pluripotent cells which promoter sequence regulates the expression of the first selectable or detectable marker gene; (ii) a second DNA construct that contains a regulatory sequence (promoter) which is selectively or preferentially expressed by cells that differentiate into a desired cell type, wherein such promoter sequence regulates the expression of said second selectable and/or detectable marker gene; and (iii) a third DNA construct that contains a constitutive promoter that regulates the expression of a third selectable or marker gene as well as methods for the production of such chimeric embryos.

It is another object of the invention to implant such chimeric embryos into the uterus of a suitable non-human recipient animal and to allow such embryo to develop into a fetus or animal containing the desired (target) differentiated cells or tissues.

It is another object of the invention to use such chimeric fetus or animal for the isolation of the desired (target) differentiated cells, tissues, or organs.

It is another object of the invention to use such isolated cells, tissues or organs for cell therapy, gene therapy and/or transplantation therapies.

It is another object of the invention to use such chimeric animals or fetuses as animal models for the study of potential therapeutics (e.g., cells may further contain an oncogene or virus) or for cell differentiation studies.

It is yet another object of the invention to use the resultant differentiated cells, tissues, or organs derived from such chimeic fetuses or animals for the production of pharmaceuticals, cell differentiation studies, and/or as donor cells for nuclear transfer.

### BRIEF DESCRIPTION OF THE FIGURES

FIG. 1 depicts schematically the transfection or transformation of a non-humam primate (Cynomolgus monkey) ES or ES-like cell with a DNA construct that comprises three transgenes respectively comprising different promoters and selectable or detectable markers which are expressed under the regulatory control of these promoters and which are expressed under regulatable or constitutive conditions.

FIG. 2 depicts schematically the selection of the transgenic monkey ES cells which have been transfected with the DNA construct containing the three transgenes depicted in FIG. 1.

FIG. 3 depicts schematically the introduction of transgenic non-primate (Cynolmolgus monkey cell) transfected with a DNA construct containing the three transgenes in FIG. 1 and selected as shown in FIG. 2 into a mouse embryo to produce a chimeric mouse/monkey embryo, transfer thereof into the uterus of a female recipient mouse which is maintained initially under one st of selection conditions (permiting survival of primordial germ cells (PGCs) and thereafter maintained under a second set of selection conditions that permits the selective or preferential survival of desired fetal monkey differentiated cells (monkey oocytes exemplified), and which chimeric fetus is permitted to give rise to a chimeric animal (mouse) that expreses the desired human or non-humam primate (monkey) differentiated cells (monkey oocytes).

### SUMMARY OF THE INVENTION

This invention relates to methods for the efficient production of desired (target) differentiated cell types from embryonic stem or stem-like cells, preferably primate embryonic stem cells. Differentiated cells produced by the present methods may be used e.g., for cell therapy, the production of pharmaceuticals, drug studies, cell differentiation studies and the like.

Prior to escribing the invention in further detail, the folowing definitios are provided. Otherwise all terms in this application are to accorded their ordinary meaning as they would be construed by a skilled person in the field of stem cells, embryo transfer techniques, cloning and/or nuclear transfer technology.

“Pluripotent cells” or “Pluripotent stem cells” or “embryonic stem cells” (“ES cells”) and “primordial germ cells” (PGC's) are pluripotent cells which may be derived from pre-embryonic, embryonic, or fetal tissue at any time after fertilization, which possess the characteristic of being capable under appropriate conditions of producing progeny of several different cell types that are derivatives of the three germinal layers (endoderm, mesoderm and ectoderm) according to standard art-recognized tests such as the ability to form teratomas in a 8-12 week SCID mouse. Pluripotent cells or pluripotent stem cells or ES cells as defined herein specifically includes cells derived from embryos produced by natural fertilization and in vitro fertilization techniques as well as embryos produced by nuclear transfer methods and parthenogenically activated embryos such as the methods described in the Robl patent application incorporated by reference herein.

Further, expressly included in this definition are non-human and human primate embryonic cells, as exemplified by monkey stem cells produced by parthenogenic embryos, human ES cells as described by Thomson et al., Science,228:1145(1998); stem cells from Rhesus monkeys as described in Thomson et sl., Biol. Reprod. 55:254 (1996); marmoset stem cells, (Shamblott et al., Proc. Natl. Acad. Sci., USA 95:13726 (1998)). Any type of pluripotent cell, particularly pluripotent primate cells that are capable of producing progeny which include all three lineages are included in the terms defined herein.

“Feeder cells” is intended to refer to the cells that are typically used to co-culture such pluripotent cells in culture and include by way of example primary mouse embryonic fibroblasts, immortalized mouse embryonic fibroblasts, human fibroblast-like cells derived from human ES cells, STO cells and the like. [Alternatively, such pluripotent cells may be cultured in the absence of feeder cells if appropriate growth factors, hormones, etc. are present as is generally known in the stem cell art.]

“Embroid bodies” refers to aggregates of differentiated and undifferentiated cells that appear when pluripotent cells overgrow in monolayer cultures, or are maintained in suspension cultures. Embroid bodies are a mixture of different cell types, typically containing from several germ layers.

“Stem cell” can refer to either a pluripotent stem cell or a committed precursor cell. Minimally a stem cell has the ability to proliferate and form cels of more than one phenotype, and is also capable of self-renewal-either as part of the same culture, or when cultured under different conditions. Embryonic stem cells are positive for the enzyme telomerase and express OCT-4.

“Committed precursor cells”, “lineage restricted precursor cells”, and “restricted developmental lineage cells” refer to cells capable of proliferating and differentiating into several different cell types, with a range that is typically more limited than pluripotent cells of embryonic origin capable of giving rise to cells in all three germ layers. Non-limiting examples of committed precursor cells include by way of example hematoietic stem cells, hepatocyte progenitors, and mesenchymal stem cells, neural restricted cells, and neuronal precursors.

“Differentiated” cells herein generally refer to cells comprising one or more of the relatively mature phenotypes that a stem cell can generate-as discernable by morphological criteria, antigenic markers, and gene transcripts they produce. By contrast “undeifferentiated cells” herein generally refer to self-renewing stem cells or pluripotent cells as defined supra.

“Transgenic cells” herein refers to cells, prefeably stem cells into which have been introduced one or more nucleic acid sequences e.g., by transfection, infection, microinjection or transformation mthods. In the present invention stem cells are typically transfected or transformed with one or more DNA constructs that encode different selectable or detectable markers which are selectively expressed in differentiated or undifferentiated cells.

A “detectable” or “selectable” marker gene herein referes to any gene that produces a product that facilitates the selection of cells of a desired phenotype, e.g., undifferentiated cells or differentiated cells of one or more specific lineages. Examples therof include genes that neomycin phosphotransferase gene (neo), puromycin resistance gene (puro), hygromcin resistance gene (GFP), YFP and the like. Also, these markers include genes which encode readily detectable gene products such as green fluorescent protein (GFP) detectable enzymes, detectable peptides and the like. In the present invention the selectable marker facilitates the selective ablation of non-target cells or the selective survival and selection of target cells in vivo, generally in an immunodeficient non-human mammal, such as a SCID or nude mouse. Therefore, the selectable markers must be suitable for use in the recipient non-human mammal. This is preferably accomplished by using as the recipient non-human mammal one which is resistant to or not adveresly affected by the particular selectable or detectable markers. Such animals may be produced by transgenic methods or by nuclear transfer methods using donor cells that are transgenic and express desired selectable or detectable marker genes.

“Regulatory sequence” herein generally refers to a nucleic acid sequence that effects the transcription of a gene operably linked thereto. This includes constitutive and regulatable promoters. Typically these regulatory sequences will comprise mammalian promoters of genes that are preferentially expressed in undifferentiated cells or differentiated cells of a desired lineage, e.g., neural cells.

“Regulatory sequence preferentially expressed in pluripotent or undifferentiated cells” herein generally refers to promoters that are more expressed, generally at least 5-fold greater in undifferentiated cells than differentiated cells. Examples thereof include the telomerase promoter (TERT), OCT-4 promoter, SSEA-4 promoter, tra-1-60 promoter, and tra-1-81 promoter. These genes all constitute markers characteristically expressed by undifferentiated cells.

“Regulatory sequence selectively or preferentially exprssed by cells that differentiate into a desired cell type or cell types” refers to promoters of genes preferentially more expressed in a desired differentiated cell type, e.g., an oocyte, a neural cell, a muscle cell, a bone cell, an immune cell, a cardiac cell, a liver cell, and the like. Examples thereof include the Vasa gene which is “on” when monkey pluripotent cells differentiate into oocytes; genes prefentially expressd by liver cells such as alpha-fetoprotein, albumin, alpha-1 antitrypsin, glucose-6-phosphatase, cytochrome p450, transferrin, asialoglycoptotein receptor, and BNF-4 alpha; genes preferentially expressd by neural cells such as beta-tubulin EIII or neurofilament characteristic of neurons, glial fibrillary acidic protein (GFAP) present in astrocytes, galactocebroside (GAIC) or myelin basic protein present in oligodendrocytes, nestin characteristic of neural precursors, AB25 and NCAM characteristic of glial and neural progenitors; genes preferentially expressd by skeletal muscle such as myoD, myogenin and myf-5; genes preferentially expressed by endothelial cells such as PEKAM, Flk-1, tie-1, tie-2, vascular endothelial cadherin, MECA-32 and MECA-14.7; genes preferentiallly expressd by smooth muscle cells such as specific myosin heavy chain; genes preferentially expressed by cardiac cells such as GATA-4, Nkx2.5, cardiac troponin I, alpha-myosin heavy chain; genes preferentially expressed by pancreatic cells such as pdx and insulin secretion genes; genes preferentially expressed by hematoietic cells such as GATA-1, CD34, AC133, beta-major globulin, and beta-major like gene PH1 for hematoietic cells and their progenitors, and the like. Other examples of genes preferentially expressed by a desired differentiated cell type can be identified by known methodologies, e.g, immunological techniques such as the use of immuncytochemistry to detect cell surface markers, immunohistochemistry, Western blot analysis of cell extracts, ELISA, and gene expression methods that detect mRNAs such as Northern blot analysis, dot-dot-hybridization analysis, and reverse-transcriptase initiated polymerase chain reaction (RT-PCR) using sequence-specific primers.

“Regulatable promoter” refere to a promoter that is “on” under specific conditions, e.g., when a particular growth factor is present.

“Constitutive promoter” refers to a promoter that is always “on”.

“Nuclear transfer embryo” refers to an embryo obtained when a donor cell or the genetic material therefrom is “reprogrammed” by being placed in contact with cytoplasm of an undifferentiated or relatively undifferentiated cell, e.g, an oocyte, blastocyst, or a pluripotent cell and permitted to become activated such that it develops into an embro that expresses the genetic material of the donor cell. The donor cell may be of the same or different species origin relative to the cell which is the source of the reprogramming cytoplasm.

“Parthenogenic embryo” refers to the activation of male or female sex cells which have not been fertilized under conditions such that give rise under appropriate culture conditions a parthenogenic embryo containing pluripotent cells that give rise to cells of all three lineages. Typically, parthenogenic embryos are incapable of giving rise to live offspring when implanted into the uterus of a female recipient.

“Lineage defective pluripotent cell” refers to a pluripotent cell which has been genetically modified, e.g., by knock-out of one or more genes, such that it is incapable of giving rise to specific cell lineages, e.g., neural cells, cardiac cells, etc.

Thus, in its broadest embodiments, the invention provides novel in vivo methods for deriving desired (target) differentiated cell types from embryonic stem or stem-like cells, e.g., any ES or ES-like cell of mammalian origin and particularly including non-human primate, human, mouse, rat, hamster, bovine, equine, goat, sheep, canine, feline, panda, bear, guinea pig, zebra, elephant, hippopotamus, rhinoceros, and the like. However, in the preferred embodiment, the invention provides novel methods for deriving desired primate differentiated cell types from human and non-human primate embryonic stem or stem-like cells.

The embryonic stem cells used as a starting material in the invention methods may be obtained by known methods. For example, in the case of non-human primate and human ES cells, ES cell lines are commercially available. Alternatively, embryonic stem cells may be desired from non-human embryos, umbilical cord, umbilical cord blood samples, and other known sources of pluripotent stem cells. Still alternatively, embryonic stem and stem-like cells, e.g., primate embryonic stem cells may be obtained from nuclear transfer embryos and parthenogenically derived primate embryos.

It is now well known that embryonic stem cells including human and other primate embryonic stem cells are capable of differentiating into a variety of different tissues i.e., cell tissues that are present in an intact animal. This feature of embryonic stem cells has been confirmed with a variety of different types of embryonic stem cells using known in vitro and in vivo models. As noted above, a conventional in vivo model for study of the differential capacity of embryonic stem cells comprises injecting the particular ES cell into an immunodeficient (SCID) mouse and allowing the stem cells to develop into teratomas. These studies have confirmed the differentiation capacity of ES cells as the resultant teratomas may be comprised of a myriad of different adult differentiated tissues, e.g., gut, hair follicle, muscle, brain, bone, respiratory, epithalial, et al. While this is a useful means of studying tissue differentiation and generating different differentiated cell types, it is disadvantageous because it does not facilitate a systematic study of the differntiation of an embryonic stem cell inot a desired (target) differentiated cell. Because the differentiation process occurs in vivo, (in the mouse), the precise conditions that affect the differentiation process and differentiation pathways, e.g., growth factors, hormones, helper cells, transcription factors, etc., can not be regulated or controlled. Essentially, because the process occurs within the body of the mouse all the intermediate steps that control differentiation are out of reach.

By contrast, the present invention seeks to combine the intrinsic advantages of the immunodeficient mouse as a vehicle for facilitating ES differentiation into desired (target) cell types while eliminating the siginificant inherent disadvantages (random, uncontrolled cell differentiation) by the use of molecular biology and embryo transfer technology. Specifically, according to the present invention, a desired ES cell type, preferably a non-human ES cell, will be injected into an early stage embryo, typically a blastocyst or morula stage mouse embryo thereby generating a chimeric embryo, comprised of primate ES cells and mouse embryonic cells. These embryos will then be allowed to develop preferably until about 10-15 days post-conception preferably by implantation of the chimeric-embryo into a surrogate female, mouse, preferably an immunodeficient mouse so as to obviate a potential rejection response against the chimeric-embryo, and particularly the non-mouse (primate) cells that are comprised in the chimeric-embryo. After the embryos have been allowed to develop until about 10-15 days post-conception, the resultant fetuses are then isolated, euthanized, fixed and non-mouse cells, e.g., monkey cells identified using specific antibodies that screen for appropriate markers expressed by the non-mouse cells (e.g., green flurescent protein gene may be introduced into the mouse ES cells and used as a marker). The identification of non-mouse cells that express the marker will confirm that a chimeric animal which comprises mouse and non-mouse cells has been obtained. This chimeric animal therefore is a potential source of desired non-mouse (e.g., monkey) cells, e.g., monkey tissues and organs systems contained in the resultant chimeric animal. Thereby, it is possible to produce any desired primate differentiated cell type in vivo. While this outcome is itself advantageous, the present invention takes this in vivo differentiation process a step further in a process referred to by the present inventors as “differential ablation of non-desired tissues”.

Essentially, the present invention provides a mean of selectively producing desired (target) differentiated cell types, tissues and/or organs from desired ES or ES-like cells in vivo by the creation of genetically modified ES cells that have been engineered with specific DNA sequences such that when they are allowed to differentiate in a chimeric embryo only desired (target) differentiated cells are ablated.

Particularly, according to the present invention, transgenic ES or ES-like cells are created that comprise at least the following combination of DNA constructs:
(i) a DNA construct that encodes a selectable marker that provides for the selection of transgenic ES or ES-like cells;
(ii) a DNA construct that contains a selectable marker that is only expressed by transgenic ES or ES-like cells when the cells are in a pluripotent state, e.g., primordial germ cells; and
(iii) a DNA construct that encodes a selectable marker that is only expressed by transgenic pluripotent cells that differentiate into the desired (target) differentiated cell type, e.g., oocytes or another desired differentiated cell type tissue or organ e.g., neuronal cells, hepatocytes, cardiac cells, lung and other respiratory cells, esophageal cells, pancreatic islet cells, stomach and other digestive cells, tracheal cells, gall bladder cells, hematopoietic and other blood cell types, e.g., erythrocytes, T and B lymphocytes, monocytes, oligodendrocytes, retinal and other ocular cells, auditory cells, etc., and tissues and organs containing such differentiated cells.

More specifically, an ES or ES-like cell will be obtained that comprises the following DNA constructs:
(i) a first DNA construct that contains a selectable and/or detectable marker, e.g., neomycin phosphotransferase and/or green fluorescent protein the transcription of which is regulated by a promoter that is only “on” when cells are in a pluripotent state, e.g., primordial germ cells, e.g., Oct-4, TERT or another promoter that is only “on” or expressed in pluripotent cells, e.g., primordial germ cells. This selectable marker e.g., Neo (neomycin phosphotransferase) will only be expressed in pluripotent cells and will allow for these pluripotent cells selectively to survive under the appropriate selection conditions and/or will preferentially express a detectable marker, e.g., green fluorescent protein, that will be detected only in pluripotent cells, e.g., primordial germ cells because the expression thereof is regulated by a promoter, e.g., Oct-4, TERT or another promoter which is only “on” in pluripotent cells;
(ii) a second DNA construct that encodes at least one selectable marker and/or a detectable marker, the expression of which is controlled by a promoter that is only “on” in cells that differentiate into desired (target) cells, tissues or organ types, e.g., oocytes; and
(iii) a third DNA construct that contains a selectable and/or a detectable marker gene that is always “on” because the expression thereof is regulated by a constitutive promoter thereby providing for the selection of transgenic ES or ES-like cells.

After the resultant transgenic donor ES or ES-like cells are obtained that contain at least the above-described three DNA constructs, i.e., by selection for cells that express the selectable or detectable marker gene expressed by the third DNA construct, these transgenic donor ES or ES-like cells, e.g., transgenic primate ES or ES-like cells, e.g., transgenic Cynomolgus monkey ES cells will be introduced into a suitable recipient non-human mammalian embryo, e.g., a mouse embryo, that preferably is itself transgenic. Particularly, the recipient embryo will preferably contain and express at least one selectable marker expressed by the first DNA construct (that is “on” only in pluripotent cells) contained in the transgenic donor ES or ES-like cells, e.g., Neo, and will further express at least one selectable marker which is expressed by the second DNA construct contained in the transgenic donor ES or ES-like cells, e.g., a puromycin resistance marker, that is only “on” in non-host ES or ES-like cells that mature into the desired (target) non-host cells, e.g., primate oocytes.

The resultant chimeric embryos comprising the described transgenic host and non-host cells will then be transferred into the uterus of a suitable recipient female host, e.g., a female mouse that preferably also has been genetically engineered such that it is resistant to the same selectable markers, e.g., neomycin and puromycin. Thereby, this animal when exposed to selection conditions for the particular selectable markers is not adversely affected.

The resultant pregnant host animal, e.g., SCID mouse, will then initially be maintained under selective conditions for the selectable marker expressed by the first DNA construct, e.g., when a Neomycin phosphtransferase gene is selected as the detectable markr, G418 will be administered in the drinking water to the pregnant female until the time that primordial germ cells (PGC5) reach the gonads. (In the case of murines, this occurs about 10 days post-conception). Because the non-host transgenic cells express the selectable marker under the control of a promoter that is only “on” in pluripotent cells, e.g., Oct-4, only OCT-4 positive non-host cells will survive this initial selection process.

Thereafter a second selection process will be applied in order to preferentially select for transgenic donor ES or ES-like cells that have matured into the desired (target) differentiated cell. Particularly, the host and fetuses will be contacted with the second selection agent, e.g., puromycin, that is expressed by the second DNA construct only in transgenic donor cells that give rise to desired differentiated cell types, e.g., oocytes, and constitutively by the female recipient (mouse) and host embryo cells.

For example, in the case wherein the desired (target) non-host cells are oocytes, e.g., primate oocytes, puromycin will be administered in the recipient female's drinking water from about day 11 until the desired target cells are recovered.

After such selection process, if desired the chimeric fetuses can be recovered prior to being allowed to reach full-term, and the desired non-host differentiated cells, e.g., primate oocytes, recovered therefrom.

Alternatively, the chimeric fetuses are allowed to develop to term into a mature host animal, e.g., a mouse, and the desired (non-host) cells, e.g., primate oocytes recovered therefrom. For example, in the case of oocyte target cells upon puberty the F1 animals will ovulate primate (monkey) eggs.

As noted above, in the preferred embodiment the subject methods will utilize as the transgenic (non-host) ES or ES-like cells primate ES or ES-like cells, more preferably monkey ES cells, e.g., cynomulgus monkey or human ES cells. However, the subject process is applicable to any desired ES or ES-like cell starting material, e.g., all mammalian ES and ES-like cells, e.g., primate, murine, canine, feline, porcine, rabbit, bear, goat, bovine, sheep, panda, equine, guinea pig, and other mammalian ES and ES-like cells afore-mentioned.

As further noted previously, the ES starting material, e.g., primate ES or ES-like cells are obtained by known methods, e.g., isolated from embryonic, umbilical or other known sources of ES cells, commercially obtained, or may be isolated from embryos or fetuses produced by nuclear transfer cloning methods or from parthenogenically derived embryos or fetuses. For example, monkey ES-like cells can be from parthenogenically activated monkey oocytes.

The ES starting material will comprise authentic ES cells or “ES-like” cells. ES-like cells herein is intended to include ES cells which have been genetically modified e.g., such that they cannot differentiate into specific cell lineages and/or pluripotent cells obtained from parthenogenically derived embryos which because of the means they are produced may not comprise the identical pluripotency and other phenotypic properties of authentic ES cells.

The ES or ES-like cells which are used to produce chimeric embryos according to the present invention will be transgenic, i.e., they will contain at least the three DNA constructs described above. This may be accomplished by introduction of these DNA constructs into ES starting materials, e.g., via electroporation, transfection, or transformation. Alternatively, the ES cells may be derived from transgenic cells that contain these DNA constructs. For example, in the case of nuclear transfer derived ES cells the donor cells used to produce a cloned embryo or fetus may contain one or more of the above-described DNA constructs. For example, a non-somatic or somatic donor cell, e.g., fetal fibroblast, may be transfected or transformed by a vector containing one or more of the above-described DNA constructs such that ES cells derived from the resultant cloned embryo contain such DNA constructs.

These ES cells may further be genetically engineered to include other genes, e.g., genes that encode therapeutic proteins, enzymes, growth factors, differentiation factors, hormones that also may be regulated by promoters only expressed in specific differentiated cell types or which are expressed constitutively. Also, these cells may be engineered to contain a gene that is defective or lacking in a particular individual that is associated with a particular disease or condition.

The three DNA constructs that are necessary and essential to the invention have been described above and the techniques used to derive desired differentiated cells from chimeric embryos derived using transgenic ES cells containing these constructs are exemplified in the example which follows.

As discussed above, while the exemplary DNA constructs utilize OCT-4 as the promoter in the first DNA construct that is “on” when the transgenic ES cells are in a pluripotent state, the invention also embraces the use of other promoters that are selectively or preferentially expressed by pluripotent cells, e.g., primordial germ cells.

Likewise, while the invention exemplifies Vasa as the promoter that is “on” when monkey pluripotent cells mature into desired target cells (oocytes), the invention encompasses the use of any tissue-specific promoter, in one which is selectively on when desired (target) cells are produced by differentiation of the non-host (e.g., monkey) pluripotent cells, i.e., primordial germ cells.

Finally, while the invention exemplifies the use of Neo, poromycin, Hygro GFP, and YFP as suitable selectable and/or detectable marker genes, the invention embraces the use of any selectable or detectable marker that is appropriate or suitable for use in the particular recipient host female, e.g., mouse, and the non-host ES or ES-like cells, in that facilitates selection of desired (target) cells and ablation of non-desired (non-target) cells. Examples of selectable and detectable marker suitable for use in mammalian systems are numerous and well known in the art. For example green fluorescent protein (GFP) is an alternative selectable marker well utilized in mammalian cells.

While the invention has been described above, in order to further illustrate the invention in greater detail the following exemplary embodiment is provided.

### EXAMPLE

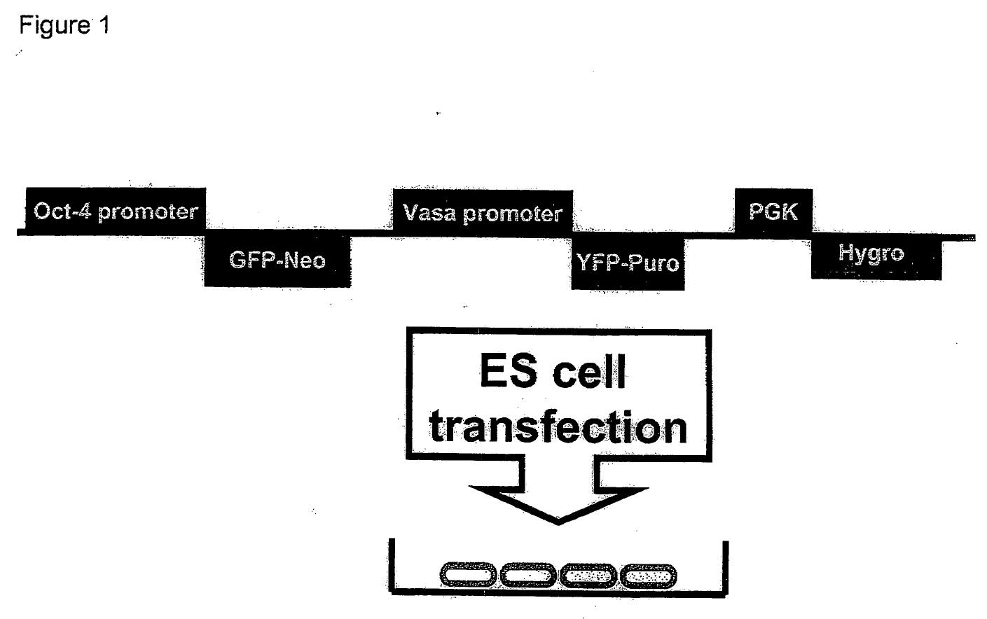

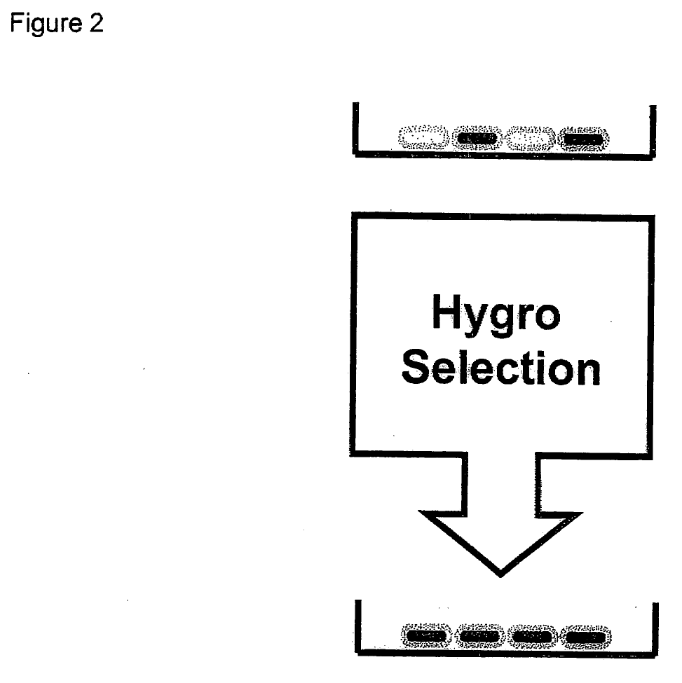

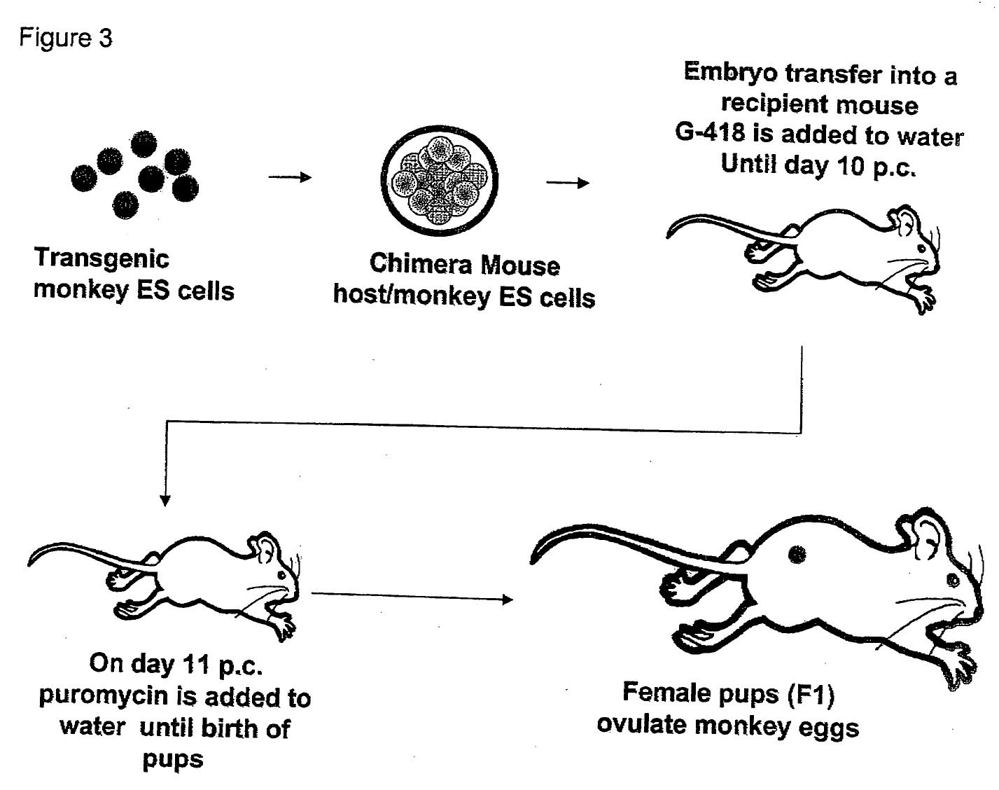

This example describes and exemplifies the inventive method of in vivo differentiation called “differentiated ablation of non-desired tissues” which yields differentiated cells of a desired lineage. Particularly, the following procedures are used in order to obtain a chimeric mouse that produces monkey oocytes:
1—Monkey ES cells are transfected with a vector that contains three different DNA fragments as shown schematically in FIG. 1 and below:
   a. Oct-4/CFP/Neo. The first DNA construct contains a promoter which is preferentially ‘on’ and regulates the expression of a first selectable marker gene in cells which are in the pluripotent state and when they are primordial germ cells;
   b. Vasa/YFP/Puromycin. The second DNA construct contains a promoter which is preferentially ‘on’ and regulates the expression of a second selectable marker gene in germ cells which are maturing from primordial germ cells into oocytes;
   c. PGK/Hygro The third DNA construct comprises a third selectable marker the expression of which is regulated by a constitutive promoter e.g., PGK-Hygro which marker genes provides a means for the selection of transgenic ES cells.
2—In the second step, monkey ES cells are injected into a host mouse embryo that is resistant to the first and second selectable markers (neomycin and to puromycin) as shown schematically in FIG. 2.
3—In the third step, the resultant chimeric embryos are transferred into the uterus of a recipient female that is also transgenic for neomycin and puromycin as shown schematically in FIG. 3.
4—Neomycin (G418) is then administered via drinking water to the pregnant female from the day she receives the embryos until the time when primordial germ cells (PGC's) reach the gonads (˜Day 10 p.c.). Only OCT-4 positive cells survive the selection process as shown schematically in FIG. 3 and described below.
   a. Puromycin is administered via drinking water at the time PGC's start to differentiate into eggs (˜Day 11 p.c.) and maintained until the fetuses are removed on day 15 p.c.
5—The fetuses are analyzed and the only monkey cells observed are eggs. Another experiment is then performed to allow the resultant fetus to develop to term and into a mature mouse. Upon puberty, F1 animals ovulate monkey eggs (FIG. 3). (This is confirmed by assaying these oocytes for the expression of monkey-specific antigens).

1. A transgenic embryonic stem (ES) or embryonic stem-like (ES-like) cell that comprises at least the following DNA constructs:
i. a first DNA construct that contains a regulatory (promoter) sequence selectively or preferentially expressed in pluripotent cells that regulates the expression of at least one selectable and/or detectable marker gene;
ii. a second DNA construct that contains a regulatory (promoter) sequence selectively or preferentially expressed by cells that differentiate into a desired cell type, which sequence regulates the expression of at least one selectable and/or detectable marker gene; and
iii. a third DNA construct that contains a selectable or detectable marker gene that is expressed under the control of a constitutive promoter.

2. The transgenic ES or ES-like cell which is selected from the group consisting of primate, equine, murine, canine, feline, bovine, goat, sheep, bear guinea pig, panda, and rabbit ES or ES-like cells.

3. The transgenic ES or ES-like cell which is primate.

4. The transgenic ES or ES-like cell of claim 3 which is a monkey ES-or ES-like cell.

5. The transgenic ES or ES-like cell of claim 1 which is obtained from an embryo.

6. The transgenic ES or ES-like cell of claim 5 wherein said embryo is obtained by nuclear transfer cloning methods.

7. The transgenic ES or ES-like cell of claim 5 wherein said embryo is obtained by parthenogenic methods.

8. The transgenic ES or ES-like cell of claim 1 which is further genetically engineered such that it is incapable of differentiating into a specific cell lineage.

9. A chimeric non-human embryo or fetus of claim 9 which is murine.

10. The chimeric non-human embryo or fetus of claim 9 which is murine.

11. A non-human recipient female that comprises a chimeric non-human embryo according to claim 9.

12. A differentiated cell, tissue or organ derived from the chimeric non-human embryo, fetus or animal of claim 9.

13. The differentiated cell, tissue or organ of claim 12 which comprises a cell selected from the group consisting of an oocyte, neural cell, hematopoietic cell, digestive cell, respiratory cell, organ cell, eye cell, ear cell, olfactory organ cell, skin cell, circulatory organ cell, reproductive organ cell, urinary organ cell, taste organ cell, and immune cell.

14. The differentiated cell, tissue or organ of claim 13 which is a monkey oocyte.

15. A method of producing a chimeric non-human embryo comprising introducing at least one transgenic ES or ES-like cell according to claim 1 into a recipient non-human embryo.

16. The method of claim 15 wherein said embryo is a blastocyst-stage embryo.

17. The method of claim 15 wherein the resultant chimeric non-human embryo is implanted into the uterus of a non-human female recipient animal.

18. The method of claim 17 wherein said animal is a mouse.

19. The method of claim 18 wherein said mouse is immunocompromised.

20. The method of claim 17 wherein said non-human chimeric embryo is allowed to develop into a fetus.

21. The method of claim 17 wherein said recipient animal is contacted with a selection agent that permits survival of cells that express the selectable marker contained on the first DNA construct which are preferentially or selectively expressed by pluripotent (primordial germ or ES cells).

22. The method of claim 21 wherein said contact is maintained until about 10 day post-conception.

23. The method of claim 22 wherein the chimeric embryo is thereafter contacted with a selection agent that permits survival of cells that express a selectable or detectable marker expressed by the second DNA construct which is only expressed by non-recipient transgenic cells which differentiate into a particular differentiated cell, tissue and/or organ.

24. The method of claim 23 wherein said differentiated cell, tissue or organ is a monkey differentiated cell, tissue or organ.

25. The method of claim 24 wherein said differentiated cell, tissue or organ is a monkey oocyte.
